SEQUENCE LISTING (26 sequences)

SEQ ID NO: 1 - protein, 14 aa, Helianthus annuus
GRCTKSIPPICFPD

SEQ ID NO: 2 - protein, 14 aa, Artificial Sequence
  SFT1 with altered moieties at position 7
GRCTKSXPPICFPD

SEQ ID NO: 3 - protein, 14 aa, Artificial Sequence
  SFTI1 with substituted amino acid on position 7
GRCTKSXPPICFPD

SEQ ID NO: 4 - protein, 14 aa, Artificial Sequence
  SFTI1 with substituted amino acid on position
GRCTKSXPPICFPD

SEQ ID NO: 5 - protein, 14 aa, Artificial Sequence
  SFTI1 with substituted amino acid on position
GRCTKSXPPICFPD

SEQ ID NO: 6 - protein, 14 aa, Artificial Sequence
  SFTI1 with substituted amino acid on position
GRCTKSXPPICFPD

SEQ ID NO: 7 - protein, 14 aa, Artificial Sequence
  SFTI1 with substituted amino acid on position
GRCTKSXPPICFPD

SEQ ID NO: 8 - protein, 14 aa, Artificial Sequence
  SFTI1 with substituted amino acid on position
GRCTKSXPPICFPD

SEQ ID NO: 9 - protein, 14 aa, Artificial Sequence
  SFTI1 with substituted amino acid on position
GRCTKSXPPICFPD

SEQ ID NO: 10 - protein, 14 aa, Artificial Sequence
  SFTI1 with substituted amino acid on position
GRCTKSIPPXCFPD

SEQ ID NO: 11 - protein, 14 aa, Artificial Sequence
  SFTI1 with substituted amino acid on position
GRCTKSIPPXCFPD

SEQ ID NO: 12 - protein, 14 aa, Artificial Sequence
  SFTI1 with substituted amino acid on position
GRCTKSIPPXCFPD

SEQ ID NO: 13 - protein, 14 aa, Artificial Sequence
  SFTI1 with substituted amino acid on position
GRCTKSIPPXCFPD

SEQ ID NO: 14 - protein, 14 aa, Artificial Sequence
  SFTI1 with substituted amino acid on position
GRCTKSIPPXCFPD

SEQ ID NO: 15 - protein, 14 aa, Artificial Sequence
  SFTI1 with substituted amino acid on position
GRCTKSIPPXCFPD

SEQ ID NO: 16 - protein, 14 aa, Artificial Sequence
  SFTI1 with substituted amino acid on position
GRCTKSIPPXCFPD

SEQ ID NO: 17 - protein, 14 aa, Artificial Sequence
  SFTI1 with substituted amino acid on position
GRCTKSIPPXCFPD

SEQ ID NO: 18 - protein, 14 aa, Artificial Sequence
  SFTI1 with substituted amino acid on position
GRCTKSIPPXCFPD

SEQ ID NO: 19 - protein, 14 aa, Artificial Sequence
  SFTI1 with substituted amino acid on position
GRCTKSIPPXCFPD

SEQ ID NO: 20 - protein, 14 aa, Artificial Sequence
  SFTI1 with Phe-Ala mutation at pos. 12
GRCTKSIPPICAPD

SEQ ID NO: 21 - protein, 14 aa, Artificial Sequence
  SFTI1 with Phe-Val mutation at pos. 12
GRCTKSIPPICVPD

SEQ ID NO: 22 - protein, 14 aa, Artificial Sequence
  SFTI1 with Phe-Ile mutation at pos. 12
GRCTKSIPPICIPD

SEQ ID NO: 23 - protein, 14 aa, Artificial Sequence
  SFTI1 with Phe-His mutation at pos. 12
GRCTKSIPPICHPD

SEQ ID NO: 24 - protein, 14 aa, Artificial Sequence
  SFTI1 with
GRCTKSIPPXCHPD

SEQ ID NO: 25 - protein, 14 aa, Artificial Sequence
  mutated SFTI1 (10Ile-Arg; 12Phe-Pro)
GRCTKSIPPRCHPD

SEQ ID NO: 26 - protein, 12 aa, Artificial Sequence
  mutated SFTI1
GRCTKSIPPRCH

(19) United States
(12) Patent Application Publication
(10) Pub. No.: US 2016/0016998 A1
(43) Pub. Date: Jan. 21, 2016

(54) HIGH-AFFINITY MATRIPTASE INHIBITORS

(71) Applicant: MERCK PATENT GMBH, Darmstadt (DE)

(72) Inventor: Harald KOLMAR, Muehltal-Trautheim (DE)

(73) Assignee: Merck Patent GmbH, Darmstadt (DE)

(21) Appl. No.: 14/760,068

(22) PCT Filed: Jan. 9, 2014
(86) PCT No.: PCT/EP2014/000021

§ 371 Date: Jul. 9, 2015

(30) Foreign Application Priority Data
Jan. 9, 2013 (EP) 13000083.9
Jan. 22, 2013 (EP) 13000310.6

(51) Int. Cl. C07K 7/08

(52) U.S. Cl. CPC C07K 7/08; A61K 38/00

(57) ABSTRACT

The present invention relates to novel matriptase inhibitors.

The present invention relates to novel, highly-potent peptidic inhibitors of the trypsin-like serine protease matriptase. Preferred molecules of the invention SDMI-1 and 2 are potent inhibitors of the pharmaceutically relevant protease matriptase at a near physiological pH and, thus, may find applications in therapy or diagnostics.

Trypsin is one of the most prominent digestive enzymes ubiquitously found in the small intestine of vertebrates.[1] Its intriguing molecular framework includes the famous catalytic triad Asp-His-Ser as a core feature implementing its proteolytic activity.[2] This prototypic architecture and the ability to cleave peptide bonds after basic residues constitutes the structural and functional groundwork of a whole class of biocatalysts referred to as trypsin-like serine proteases.[3] Members of this enzyme family are involved in diverse biological processes and occur in soluble form or as membrane-anchored entities.[4] Type II transmembrane serine proteases (TTSP), for instance, are bound to the cell surface via the N-terminus and have been characterized as important mediators of the pericellular procession and activation of various effector molecules.[3-5] Active forms of peptide hormones, growth and differentiation factors, receptors, enzymes, and adhesion molecules are generated from inactive precursors through endoproteolytic cleavage by specific TTSPs.[4] Hence, they play crucial roles in the cellular development and maintenance of homeostasis.[3]

A well-studied example of a membrane-anchored trypsin-like serine protease with pharmaceutical relevance is matriptase.[6] It is widely expressed on the surface of epithelial cells in healthy tissue where its proteolytic activity is precisely regulated by natural protease inhibitors like the hepatocyte growth factor inhibitor-1 and 2 (HAI-1, HAI-2).[6c, 6f] However, dysregulations of this physiological inhibitor-protease balance are believed to facilitate pathological processes. Indeed, a number of studies associate matriptase overexpression with the development and progression of epithelial tumors, as well as osteoarthritis and atherosclerosis.[6g-k] Furthermore, Napp et al. observed pronounced in vivo matriptase activity in a murine orthotopic pancreatic tumor model and showed that the administration of active-site inhibitors significantly reduces proteolysis of the substrate analyte.[6d] Hence, potent and selective matriptase inhibitors are of great therapeutic importance, and their development is a challenging task. To date, a number of small synthetic organic compounds as well as large antibody fragments exhibiting single-digit nanomolar to subnanomolar inhibition constants have been reported.[7]

The present inventors used the rigid and well-defined tetradecapeptide framework of the sunflower trypsin inhibitor-1 (SFTI-1) as a starting point for structure-guided lead compound optimization. This molecular scaffold possesses a canonical (substrate-mimicking) loop for anti-proteolytic activity and has already been successfully used for the generation of inhibitors of chymotrypsin, elastase, cathepsin G, β-tryptase, proteinase K or kallikrein-related peptidase.^{[10]} Additionally, a latent inhibitory activity against matriptase in the nanomolar range (Kᵢ=1-150 nM) has been reported for the bicyclic peptide at the pH optimum of the TTSP (pH ˜8.5-9).^{[11]} Very recently, we showed that this moderate potency is essentially decreased in an environment with near physiological parameters (Kᵢ=1.1 μM at pH 7.6). This is of particular importance for potential in vivo applications. A therapeutic/diagnostic agent must possess high activity under homeostasis or—in case of tumor as well as inflammation-induced hypoxia—more acidic conditions.^{[12]}

Using a structure-guided incremental optimization strategy we were able to generate SFTI-1 derivatives with single-digit nanomolar Kᵢ as well as an improved trypsin/matriptase selectivity profile.

TABLE 1
Determined inhibition constants of compounds 1-22 against
matriptase at pH 7.6 and calculated differences in free energies
of binding/dissociation compared to SFTI-1[1, 14].

| | | | | Δ_{B}G₍ₓ₎ − |
|---|---|---|---|---|
| | | | Relative | Δ_{B}G_{(SFTI-1[1, 14])}^{[c]}/ |
| Entry | | Kᵢ^{[a]}/nM | Activity^{[b]} | (kJ · mol⁻¹) |
| SFTI-1[1, 14] | | 703 ± 87^{[d]} | 1 | 0 |
| Position | 1 | 4892 ± 663 | 7.0 | −4.8 ± 0.5 |
| 7^{[e]} | 2 | 3822 ± 494 | 5.4 | −4.2 ± 0.4 |
| | 3 | 13886 ± 1711 | 19.8 | −7.4 ± 0.4 |
| | 4 | 2380 ± 291 | 3.4 | −3.0 ± 0.4 |
| | 5 | 1629 ± 200 | 2.3 | −2.1 ± 0.4 |
| | 6 | 10857 ± 1349 | 15.4 | −6.8 ± 0.4 |
| | 7 | 3342 ± 414 | 4.8 | −3.9 ± 0.4 |
| | 8 | 1252 ± 155 | 1.8 | −1.4 ± 0.4 |
| Position | 9 | 148 ± 19 | 0.21 | 3.9 ± 0.4 |
| 10^{[e]} | 10 | 513 ± 66 | 0.73 | 0.8 ± 0.4 |
| | 11 | 46287 ± 5728 | 65.8 | −10.4 ± 0.4 |
| | 12 | 8096 ± 1011 | 11.5 | −6.1 ± 0.4 |
| | 13 | 208 ± 27 | 0.30 | 3.0 ± 0.4 |
| | 14 | 2224 ± 277 | 3.2 | −2.9 ± 0.4 |
| | 15 | 556 ± 70 | 0.79 | 0.6 ± 0.4 |
| | 16 | 4750 ± 585 | 6.8 | −4.7 ± 0.4 |
| | 17 | 232 ± 29 | 0.33 | 2.7 ± 0.4 |
| | 18 | 50.6 ± 6.5 | 0.072 | 6.5 ± 0.4 |
| Position | 19 | 1614 ± 206 | 2.3 | −2.1 ± 0.4 |
| 12^{[e]} | 20 | 580 ± 72 | 0.83 | 0.5 ± 0.4 |
| | 21 | 319 ± 40 | 0.45 | 2.0 ± 0.4 |
| | 22 | 206 ± 27 | 0.29 | 3.0 ± 0.5 |

^{[a]}Determined as described in the Experimental Section.
^{[b]}Relative activity given as the ratio Kᵢ of the corresponding compound/Kᵢ of SFTI-1[1, 14].
^{[c]}Δ_{B}G₍ₓ₎ refers to the free energy of binding/dissociation of compound x. Δ_{B}G were calculated from respective Kᵢ using Δ_{B}G = −RTInKᵢ.^{[14]} Errors of the given differences Δ_{B}G₍ₓ₎ − Δ_{B}G_{(SFTI-1[1, 14])} were calculated by propagation of errors (see Supporting Information).
^{[d]}As published before.
^{[e]}The modified position of the SFTI-1[1, 14] framework is given.

TABLE 2
Determined inhibition constants of compounds 42-44 against matriptase
at pH 7.6 (8.5), calculated differences in free energies of binding/dissociation
compared to SFTI-1[1,14] as well as trypsin affinity and selectivity at pH 7.6.^{[a]}

| | | | | Sum of | | |
|---|---|---|---|---|---|---|
| | | | Δ_{B}G₍ₓ₎- | Increment | | |
| | Matriptase | Relative | Δ_{B}G_{(SFTI-1[1,14])}/ | Contributions^{[c]}/ | Trypsin | |
| Entry | Kᵢ/nM^{[b]} | Activity | (kJ · mol⁻¹) | kJ · mol⁻¹ | Kᵢ/nM | Selectivity^{[d]} |
| SFTI-1[1,14] | 703 ± 87^{[e]} | 1 | 0 | n.a. | 0.21 ± 0.03^{[e]} | 0.0003 |
| 42 | 89.5 ± 11.1 | 0.13 | 5.1 ± 0.4 | 6.9 ± 0.6 | 9.2 ± 2.1 | 0.1 |
| 43 | 11.0 ± 1.4 | 0.016 | 10.3 ± 0.4 | 9.6 ± 0.6 | 11.2 ± 2.0 | 1.0 |
| (SDMI-1) | (1.1 ± 0.3) | | | | | |
| 44 | 6.2 ± 1.2 | 0.009 | 11.7 ± 0.6 | n.a. | 38.0 ± 11.1 | 6.1 |
| (SDMI-2) | (2.3 ± 0.8) | | | | | |

^{[a]}Experiments were performed as described in the footnote of Table 1 and in the Experimental Section.
^{[b]}Values in brackets correspond to Kᵢ determined at pH 8.5.
^{[c]}Errors were calculated by propagation of errors.
^{[d]}Calculated as the ratio of Kᵢ against matriptase (pH 7.6)/Kᵢ against trypsin (pH 7.6).
^{[e]}As published before.

Combination of the most favorable amino acid substitutions and subsequent C-terminal truncation yielded SFTI-1 derived matriptase inhibitors-1 and 2 (SDMI-1 and 2) with inhibition constants in the low nanomolar to single-digit nanomolar range. The structure-guided design of the initial peptide/peptidomimetic library as well as the shortening of the amino acid sequence were inspired by in silico experiments which were used as an idea generator towards beneficial modifications of the wild-type compound.[6e] Thus, an inhibitor possessing only twelve residues as well as an inverted trypsin-matriptase selectivity in favor of the latter enzyme was developed.

Although the applied synthetic strategy using “copper-click” chemistry was applicable for the assembly of the majority of the triazolyl-containing peptidomimetics 1-16, it was incompatible with the use of non-natural amino acid azidoalanine at position 10 (compounds 9, 11, 13, and 15). In our previous works, we did not observe significant limitations using Fmoc-Aza-OH as a building block for microwave assisted Fmoc-SPPS.[15] However, the unexpected restricted applicability of the unprotected azide functionality under the described conditions was circumvented through a modified synthetic route.

Design and synthesis of a small compound library comprising SFTI-1[1,14] derivatives 1-22:

A)

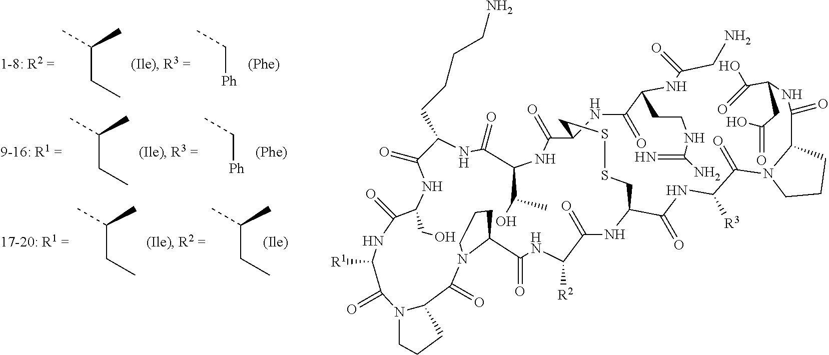

| R¹ | n | Entry | R² | n | Entry | R³ | Entry |
|---|---|---|---|---|---|---|---|
| 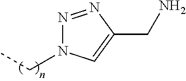 | 1 2 | 1 2 | 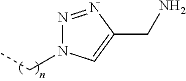 | 1 2 | 9 10 | 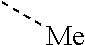 | 17 |
| 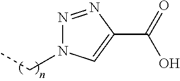 | 1 2 | 3 4 | 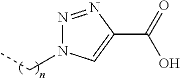 | 1 2 | 11 12 | 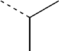 | 18 |
| 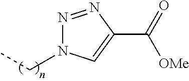 | 1 2 | 5 6 | 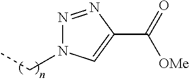 | 1 2 | 13 14 | 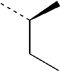 | 19 |
| 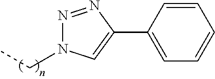 | 1 2 | 7 8 | 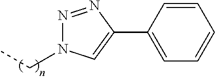 | 1 2 | 15 16 | 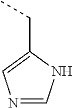 | 20 |

B)

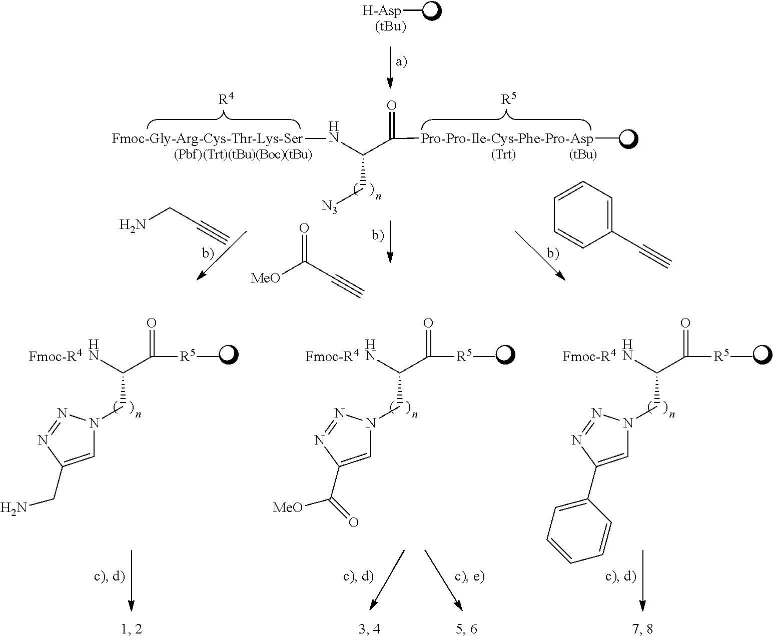

C)

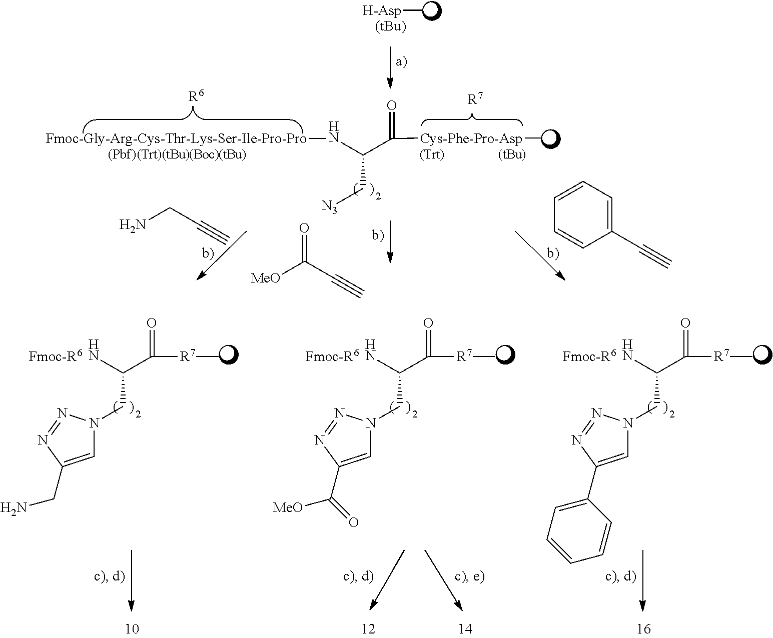

| | |
|---|---|
| 27, 30, 33: | R⁴ = Gly-Arg(Pbf)-Cys(Trt)-Thr(tBu)-Lys(Boc)-Ser(tBu), |
| | R⁵ = Pro-Pro-Ile-Cys(Trt)-Phe-Pro-Asp(tBu), n = 1 |
| 28, 31, 34: | R⁴ = Gly-Arg(Pbf)-Cys(Trt)-Thr(tBu)-Lys(Boc)-Ser(tBu) |
| | R⁵ = Pro-Pro-Ile-Cys(Trt)-Phe-Pro-Asp(tBu), n = 2 |
| 29, 32, 35: | R⁴ = Gly-Arg(Pbf)-Cys(Trt)-Thr(tBu)-Lys(Boc)-Ser(tBu)-Ile-Pro-Pro |
| | R⁵ = Cys(Trt)-Phe-Pro-Asp(tBu), n = 2 |

D)

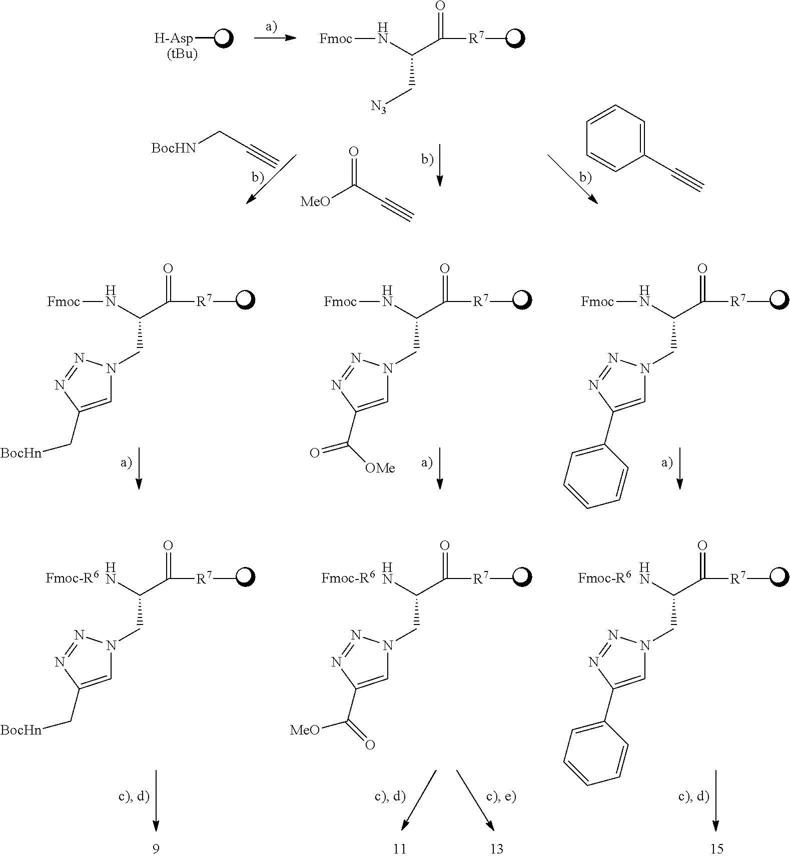

| | |
|---|---|
| 36-38: | R⁵ = Cys(Trt)-Phe-Pro-Asp(tBu) |
| 39-41: | R⁴ = Gly-Arg(Pbf)-Cys(Trt)-Thr(tBu)-Lys(Boc)-Ser(tBu)-Ile-Pro-Pro |
| | R⁵ = Cys(Trt)-Phe-Pro-Asp(tBu) |

Design and synthesis of a small compound library comprising SFTI-1[1,14] derivatives 1-22. A) Collection of triazolyl-containing peptides 1-16 as well as variants 17-22 with a singular substitutions at positions 10 and 12 using canonical amino acids lysine, argenine, alanine, valine, isoleucine, or histidine. B) Synthesis of precursors 23,24,25, and 26 as well as inhibitors 17-22. C) and D) Schematic depictions of synthetic routes yielding triazolyl-containing peptides 1-16. Conditions: a) microwave-assisted Fmoc-SPPS; b) acidolytic cleavage from the solid support using TFA/H₂O/anisole/TES (47:1:1:1, v:v:v:v) and dithiothreitol (DTT), c) air-mediated oxidative macrocyclization in 100 mM (NH₄)₂CO₃ aq (pH=8.4) at 1 mg peptide/mL; d) on-resin CuAAC using given alkyne component (5 eq), CuSO₄.5H₂O (20 mol %), sodium ascorbate (20 mol %), and N,N-diisopropylethylamine (DIEA, 8 eq) in DMF at ambient temperature (overnight) e) air-mediated oxidative macrocyclization in 100 mM (NH₄)₂CO₃ aq (pH=7.7) at 1 mg peptide/mL. All compounds 1-22 were isolated through preparative reversed-phase HPLC.

The following compounds represent preferred embodiments of the present invention, with compounds 43 and 44 being mostly preferred.

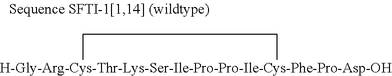

Substitutions at Position 7 (R1), 10 (R2) and 12 (R3)

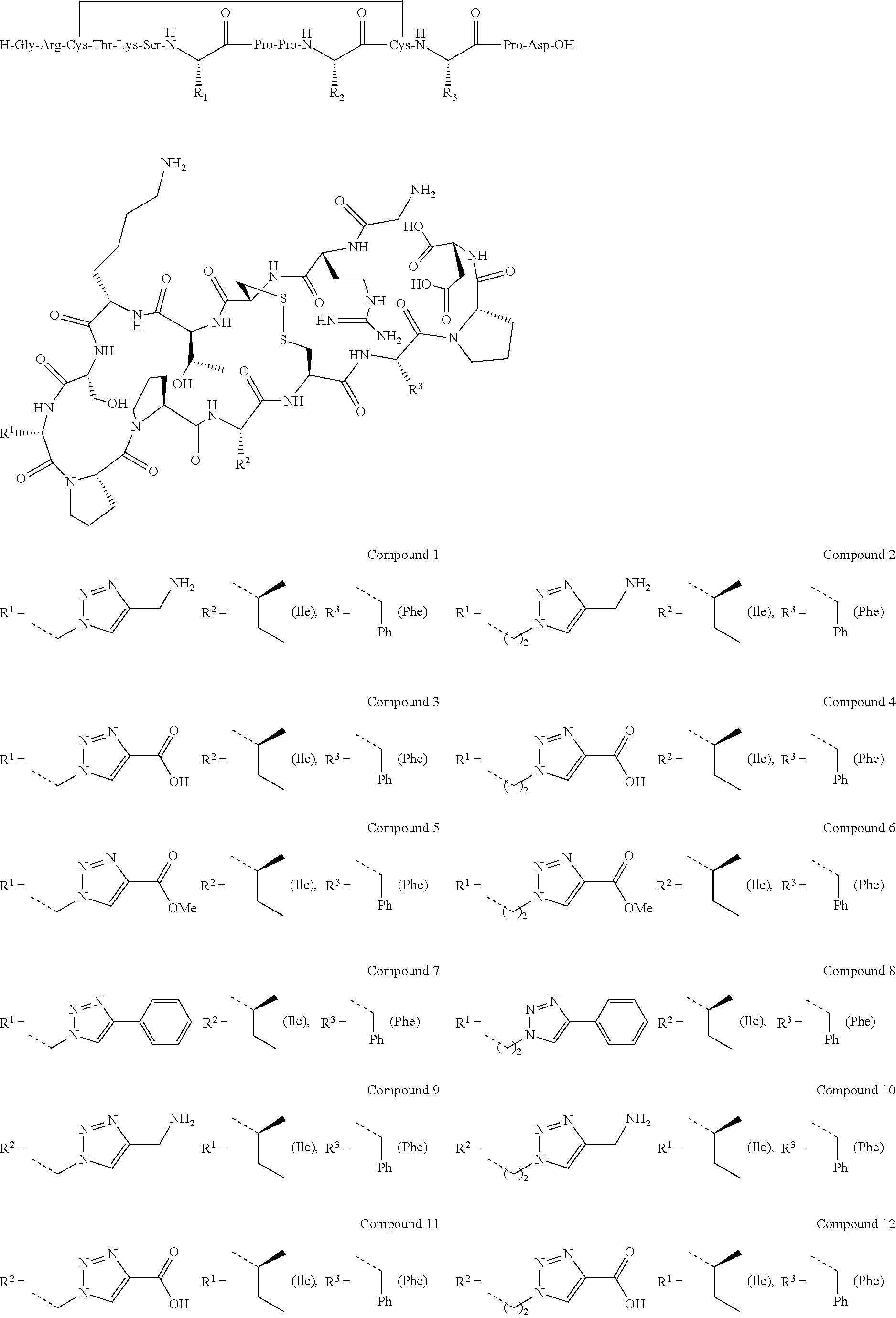

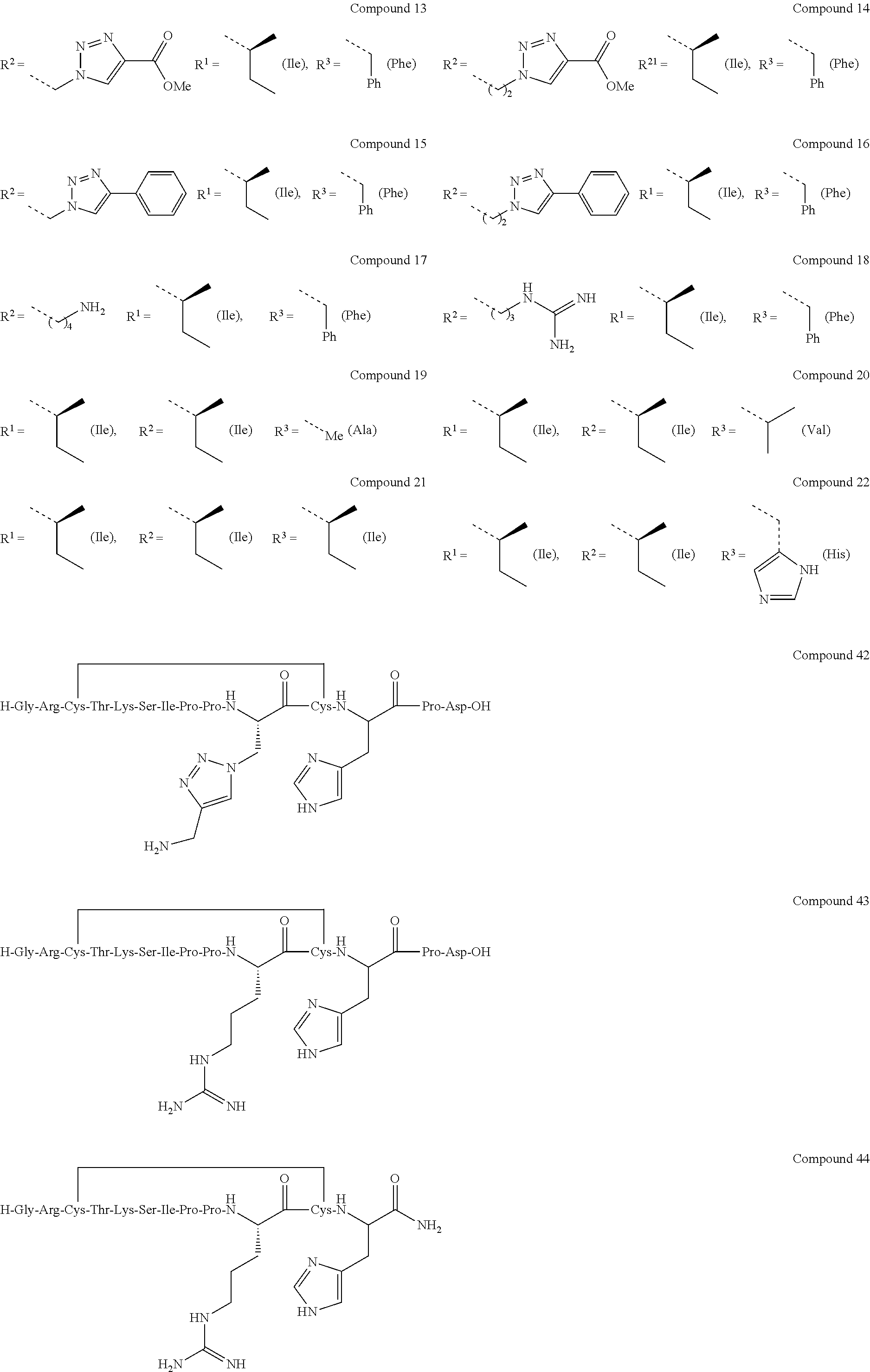

Novel compounds 43 and 44 are surprisingly potent inhibitors of matriptase in a pH range near the physiological one and, thus, may find applications in therapy or diagnostics.
Therefore, preferred embodiments of the present invention are compounds of the formula I

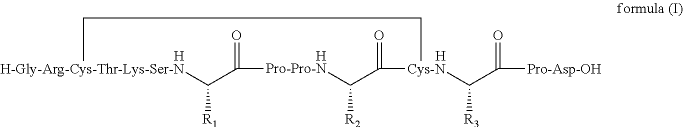

in which
R₁ denotes A or (CH₂)ₙHet,
R₂ denotes A, (CH₂)ₙHet or (CH₂)₃NHC(═NH)NH₂,
R₃ denotes A, (CH₂)ₙHet or (CH₂)ₙAr,
A denotes unbranched or branched alkyl with 1, 2, 3, 4, 5 or 6 C-atoms,
Ar denotes phenyl,
Het denotes furyl, thienyl, pyrrolyl, imidazolyl, pyrazolyl, oxazolyl, isoxazolyl, oxadiazolyl, thiazolyl, triazolyl or tetrazolyl, each of which is unsubstituted or monosubstituted by (CH₂)ₙNH₂, COON, COOA or Ar,
n denotes 0, 1, 2 or 3,
and pharmaceutically acceptable solvates, salts, tauto [symbol] mers and stereoisomers thereof, including mixtures thereof in all ratios.
Specially preferred embodiments are compounds of formula 1 in which Het denotes triazolyl or imidazolyl, which is unsubstituted or monosubstituted by (CH₂)ₙNH₂, COOH, COOA or Ar, and pharmaceutically acceptable solvates, salts, tauto [symbol] imers and stereoisomers thereof, including mixtures thereof in all ratios.
Further embodiments are compounds according to Formula (I) with non-naturally occurring substitutions at positions R₁, R₂ and/or R₃.
A specially preferred embodiment is a mutated SFTI according to formula 1, wherein R₁, R₂ and/or R₃ are selected from C₁-C₄ lower alkyl or arylalkyl.
Preferred are the following compounds, specially 42, 43 and 44, and the best embodiments are 43 and 44.
These compounds are useful in a pharmaceutical composition comprising a mutated SFTI of the invention and a pharmaceutical acceptable excipient.
Such pharmaceutical composition as are useful for the treatment of cancer.
Further preferred uses of such pharmaceutical composition comprise the treatment of a matriptase protease dysfunction related disease, wherein the disease is selected from cancer, chronic obstructive pulmonary disease, a disorder of the peripheral or central nervous system or a cardiovascular disorder, whereby symptoms of the matriptase dysfunction related disease are ameliorated.

### Step 1: Tuning Affinity of Singular Residues (Increments)

Trypsin and matriptase share a very similar substrate spectrum. Thus, we rationalized that substitutions within the canonical region of SFTI-1[1,14] would be detrimental towards improved affinity. However, we included the P2′ position (Ile7) as a site for side-chain replacements within our molecular design. Histidines 40 and 143 of matriptase are in close proximity to this residue, therefore they might provide the possibility for favourable hydrogen bonding interactions with the ligand.

To reduce the synthetic expense and to cover an adequate structural and functional space, we set up a divergent synthetic procedure. Azide-bearing peptidic scaffolds 23 and 24 were assembled on the solid support using commercially available building blocks Fmoc-L-azidoalanine (Fmoc-Aza-OH) and Fmoc-L-azidohomoalanine (Fmoc-Aha-OH). On-resin copper(I)-catalyzed azide-alkyne cycloaddition (CuAAC) with different alkyne components allowed for the facile installation of an amine, a carboxylic acid, the corresponding methyl ester and a phenyl functionality at position 7. This combinatorial approach is quite similar to the “tethered fragment” strategy previously described by Burk and coworkers.^{[13]} Acidolytic cleavage from the solid support, oxidative macrocyclization and chromatographic isolation gave SFTI-1[1,14] derivatives 1-8. Interestingly, both free carboxylic acids 3 and 4 as well as corresponding methyl esters 5 and 6 were selectively accessible from the respective open-chain methyl ester precursor peptides through the choice of macrocyclization conditions. The methyl carboxylate was hydrolyzed during disulfide bond formation at pH 8.4 giving free acids 3 and 4. Setting the pH to 7.7, however, allowed for the preservation of the methyl ester group yielding the corresponding cystine-bridged products 5 and 6.

Unfortunately, none of the position 7 variants 1-8 showed an improved matriptase affinity over SFTI-1[1,14] in enzyme inhibition assays (Table 1). Thus, we focused our further experiments on the optimization of positions 10 and 12.

In the ligand-receptor complex, the side chain of residue 10 of SFTI-1[1,14] is oriented towards a surface area of matriptase with a pronounced negative polarization. The β-carboxylic group of the Asp96 contributes significantly to this environment. Yuan et al. suggested installing short basic side chain functionalities with low degrees of conformational freedom like aminoalanine or aminohomoalanine at position 10 of the inhibitor to establish favorable electrostatic interactions.^{[6e]} Nevertheless, initial molecular modeling implied that a linker of about four to five methylene units is needed to position a basic functionality in proximity to the β-carboxylic group of Asp96 in its native conformation present in the crystal structure (PDB-ID: 3P8F). Thus, we designed SFTI-1[1,14] derivatives 9-16 possessing planar and rigid triazolyl linkers of appropriate length to investigate the possibility for new favorable interactions between the ligand and the receptor.

Initially, synthetic routes for 9-16 were devised as modifications of to the approach used for SFTI-1[1,14] variants 1-8. While variants 10, 12, 14, and 16 were readily accessible via this strategy, compounds 9, 11, 13, and 15 could not be synthesized in this manner. An attempt to assemble the Aza10 analog of peptide-resin 25 on the solid support resulted in an undefined mixture of side products (data not shown). Nevertheless, we were able to synthesize compounds 9, 11, 12, and 15 via the resin-bound pentapeptide intermediate 26 as depicted in.

Interestingly, a significant increase of matriptase affinity over SFTI-1[1,14] was observed for four of the eight triazolyl-containing peptidomimetics 9-16 (Table 1). The most pronounced enhancement within the set of inhibitors 9, 10, 13, and 15 was detected for the aminomethyl-functionalized 1,2,3-triazole 9. The difference of the free energies of binding/dissociation Δ_{B}G between compound 9 and SFTI-1[1,14] was calculated as 3.9 kJ/mol. Thus, additional favorable electrostatic inhibitor-enzyme interactions were successfully established by furnishing the peptide with a basic functionality at position 10. However, if the length of the triazolyl linker is increased by only one methylene unit (10), the attractive contribution is significantly reduced. Furthermore, the installation of an acidic carboxy group at this position (11, 12) is detrimental for matriptase inhibition. Noteworthy, a significant increase of inhibitor potency compared to SFTI-1[1,14] is also observed for the formally uncharged methyl ester derivative 13 although not as pronounced as in the case of the amino-functionalized variant 9.

Additionally, we investigated the impact of basic canonical amino acids, lysine and arginine, at position 10 (17, 18) in matriptase affinity. Surprisingly, compound 18 equipped with a flexible aliphatic linker demonstrated the highest potency of SFTI-1[1,14] variants 1-22 listed in Table 1. Due to the inherent degrees of conformational freedom of the respective amino acid side chains, a pronounced entropic penalty has been expected for compounds 17 and 18.^{[6e]} Indeed, ε-amino-functionalized derivative 17 was slightly less potent than inhibitor 9 possessing a more rigid and restrained linker motif of comparable length. However, a double-digit nanomlar Kᵢ corresponding to an increase of Δ_{B}G by 6.5 kJ/mol resulted from a singular amino acid substitution (18).

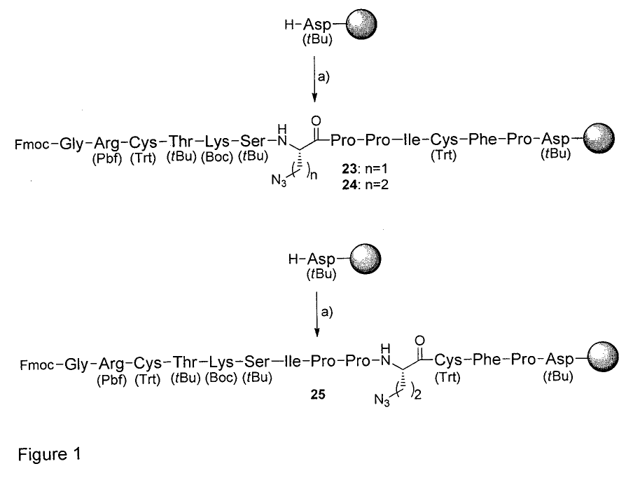

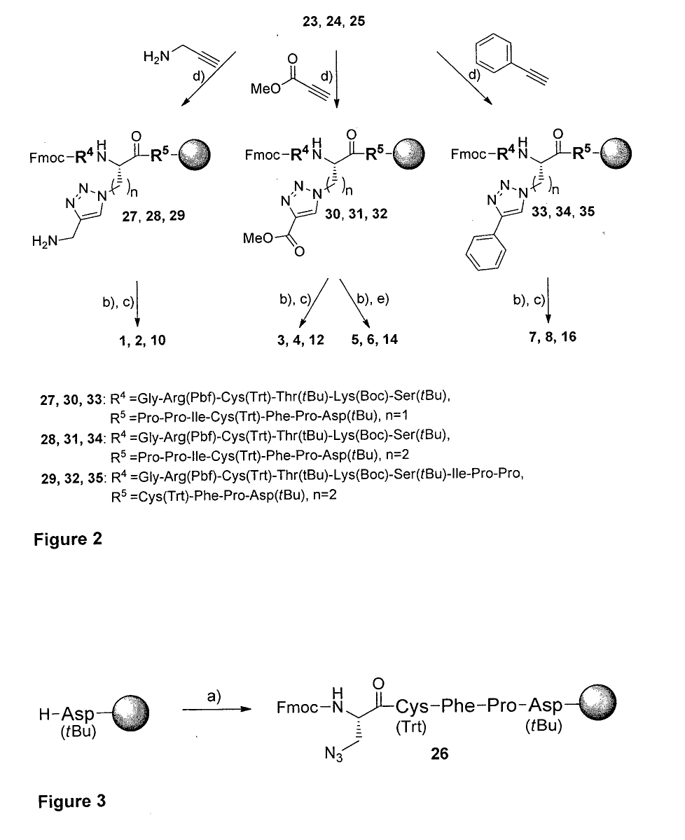

Finally, we investigated the impact of modifications at position 12 of the SFTI-1[1,14] framework in matriptase affinity. Yuan et al. described a pronounced undesirable enthropic effect on Phe12 of SFTI-1 due to conformational restraints caused by Phe97 and Phe99 side chains forming the S4 subsite of the enzyme (FIG. 1D).^{[6e]} As a consequence, substitution of Phe12 by non-natural amino acids possessing larger aromatic side chains has a detrimental effect on bioactivity.^{[11b]} Thus, we decided to replace the native phenylalanine residue by smaller aliphatic, hydrophobic, as well as heteroaromatic amino acids alanine, valine, isoleucine, and histidine (FIG. 2A). Respective compounds 19-22 were synthesized through routine microwave-assisted Fmoc-SPPS followed by oxidative macrocyclization and chromatographic isolation (see Supporting Information).

Indeed, we observed a beneficial effect of Phe12Ile (21) as well as Phe12His (22) substitutions on matriptase inhibition in our in vitro assays. In particular, the imidazole functionality at position 12 (22) facilitated an improved interaction between the SFTI-1 framework and the surface of the enzyme of about 3 kJ/mol.

### Step 2: Combination of Beneficial Increment Modifications

Encouraged by the enhanced matriptase affinities observed for compounds 9, 18 and 22, we combined the singular modifications of position 10 (9, 18) with the described Phe12His substitution (22). The resulting constructs 42 and 43 were synthesized either via microwave-assisted Fmoc-SPPS (42) or by using a strategy analogous to that described for compounds 9-16 (42). Inhibitors 42 and 43 demonstrated a further increased matriptase affinity compared to SFTI-1[1,14] derivatives 9, 18, and 22 (Table 2). Our calculations suggest that the observed improvement in Δ_{B}G relies predominantly on an additive effect. The determined individual favorable contributions of compounds 18 and 22 sum up to 9.6±0.6 kJ/mol, whereas, the difference of free energies of binding/dissociation between 43 and SFTI-1[1,14] was calculated as 10.3±0.4 kJ/mol. The difference between these two values could be accounted to a synergistic behavior.^{[14]} However, the observed effect is negligible considering the respective estimated uncertainties. In case of inhibitor 42 even an unfavorable non-additive portion was detected for the installed modifications as the calculated increase of Δ_{B}G was smaller than the sum of the incremental improvements (Table 2). Nevertheless, tetradecapeptide 43 which we refer to as the SFTI-1-derived matriptase inhibitor-1 (SDMI-1) possesses a low inhibition constant of 11 nM near the physiological pH. Furthermore, in the enzyme assay conducted near the pH optimum of matriptase (pH=8.5) a single-digit nanomolar Kᵢ (1.1 nM) was observed.

### Step 3: Truncation of Most Promising Variant

In a very recent study we performed a structural analysis of several monocyclic triazole-bridged SFTI-1 derivatives in silico. The results yielded indicated a pronounced influence of the terminal regions on matriptase affinity. An enthropic penalty caused by the fixation of the Phe12 residue of the parent bicyclic peptide through aromatic side chains of the enzyme has already been described and addressed in the presented work.^{[6e]} However, additional conformational constraints of residues Pro13 and Asp14 forming the secondary loop may cause unfavourable entropic contributions upon binding to matriptase. According to our calculations interaction with trypsin is not affected in this manner. As a consequence, the truncation of the two C-terminal residues might have a beneficial effect on matriptase inhibition. Additionally, we rationalized that removing the negative charge of the terminal carboxy group by introduction of a C-terminal amide might enhance the overall charge complementarity between the target enzyme and the inhibitor. Inspired by these considerations, we synthesized the monocyclic dodecapeptide 44 as described in the Experimental Section (Scheme 1). Indeed, we observed an additional minor improvement of inhibitor potency resulting in a K; of 6.2±1.2 nM. This SFTI-1-derived matriptase inhibitor-2 (SDMI-2) exhibits a 2.3±0.8 nM inhibition constant at pH 8.5 and shows an improved selectivity towards matriptase over trypsin (6-fold). To our knowledge, 43 and 44 are to date the most potent Bowmann-Birk matriptase inhibitors described demonstrating an inhibitory activity comparable to reported small organic compounds.^{[7a]}

Finally, we modeled the inhibitor-protease complex for SDMI-2 (44) based on the in silico coordinates of SFTI-1[1,14] and matriptase described above (see FIG. 1).[ref] First, the amino acid exchanges Ile10Arg and Phe12His were introduced into the parent model. Then, residues Pro13 and Asp14 were replaced by a C-terminal amide. Optimization of side-chain geometry and subsequent energy minimization yielded a structure quite similar to the corresponding SFTI-1 co-crystal (3P8F, FIG. 3A). Nevertheless, additional favorable interactions between 44 and matriptase were observed (FIG. 3B). The side chain of Asp96 was reoriented to form a bi-dented hydrogen bond with the guanidinyl functionality of Arg10. Furthermore, the possibility for a proton donor-acceptor interaction between the backbone carbonyl oxygen of Asp96 and the ε²-nitrogen of His12 was detected. However, an X-ray structure of the SDMI-2-matriptase complex is needed to undoubtedly prove whether the predicted in silico coordinates are valid. Yuan et al. suggested to use short basic residues like aminohomoalanine at position 10 to establish an effective electrostatic interaction with Asp96 while attenuating entropic penalties arising from extended flexible linkers.^{[6e]} However, it remains to be tested whether such a short side chain is sufficient to position a basic functionality in proximity to the carboxylic group of Asp96.

All chemicals and solvents used in the experiments that led to the present invention were purchased from Bachem, Iris Biotech, Novabiochem, Sigma-Aldrich, Rapp Polymere, Roth or Varian (Agilant).

Analytical and semi-preparative RP-HPLC were performed on a Varian 920-LC system and a Varian 940-LC system, respectively. A Phenomenex Hypersil 5u BDS C18 LC column (150×4.6 mm, 5 μm, 130 Å) and a Phenomenex Luna 5u C18 LC column (250×12.20 mm, 5 μm, 100 Å) were used as stationary phases. The eluent system consisted of eluent A (0.1% aq. TFA) and eluent B (90% aq. acetonitrile containing 0.1% TFA).

ESI mass spectra were recorded with a Shimadzu LCMS-2020 using a Phenomenex Jupiter 5u C4 LC column (50×1 mm, 5 μm, 300 Å) as well as a binary eluent system consisting of eluent A (0.1% aq. formic acid, LC-MS grade) and eluent B (100% acetonitrile containing 0.1% formic acid, LC-MS grade).

Peptides were synthesized using a Liberty 12-channel automated peptide synthesizer on a Discover SPS microwave peptide synthesizer platform (CEM) following the Fmoc strategy.

FIG. 1 shows microwave assisted Fmoc-SPPS Synthesis of precursor scaffolds 23, 24,25.

FIG. 2 shows d) on-resin CuAAC using given alkyne component (5 eq), CuSO₄.5H₂O (20 mol %), sodium ascorbate (20 mol %), and N,N-diisopropylethylamine (DIEA, 8 eq) in DMF at ambient temperature (overnight) b) acidolytic cleavage from the solid support using TFA/H₂O/anisole/TES (47:1:1:1, v:v:v:v) and dithiothreitol (DTT), c) air-mediated oxidative macrocyclization in 100 mM (NH₄)₂CO₃ aq (pH=8.4) at 1 mg peptide/mL. e) air-mediated oxidative macrocyclization in 100 mM (NH₄)₂CO₃ aq (pH=7.7) at 1 mg peptide/mL.

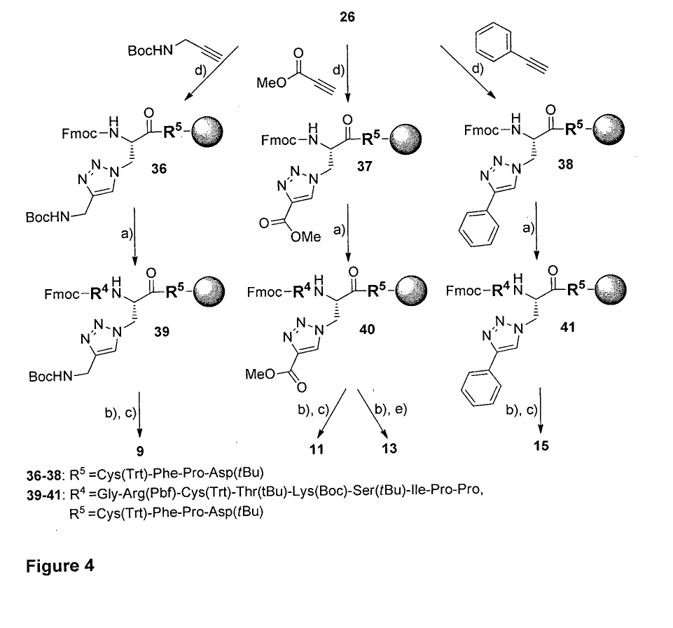

FIG. 3 shows a) microwave-assisted Fmoc-SPPS. Synthesis of scaffold 26. FIG. 4 shows synthesis of 9, 11, 13, 15. d) on-resin CuAAC using given alkyne component (5 eq), CuSO4.5H2O (20 mol %), sodium ascorbate (20 mol %), and N,N-diisopropylethylamine (DIEA, 8 eq) in DMF at ambient temperature (overnight) a) microwave-assisted Fmoc-SPPS. b) acidolytic cleavage from the solid support using TFA/H₂O/anisole/TES (47:1:1:1, v:v:v:v) and dithiothreitol (DTT), c) air-mediated oxidative macrocyclization in 100 mM (NH₄)₂CO₃ aq (pH=8.4) at 1 mg peptide/mL. e) air-mediated oxidative macrocyclization in 100 mM (NH₄)₂CO₃ aq (pH=7.7) at 1 mg peptide/mL. On-support copper(I)-catalyzed azide-alkyne cycloaddition (CuAAC) was conducted with 0.05 mmol of intermediates 26 and the respective alkyne component (N-Boc-propargylamine, methyl propiolate, or phenylacetylene) according to the scheme laid out in FIG. 4. A solution of 5 eq alkyne, 1 eq copper(II) sulfate pentahydrate (CuSO₄. 5 H₂O), 1 eq sodium ascorbate (NaAsc) and 8 eq DIEA in 5 mL argon-flushed DMF was added to the peptide resin and shaken at ambient temperature overnight. Then, the solution was removed by filtration and the peptide resin was washed with methanol (3×), 0.5% sodium diethyldithiocarbamate in DMF (w/v, 3×), DMF (3×) and DCM (3×) yielding intermediates 36-38. The Fmoc protecting group was removed according to the described procedure. The remaining N-terminal residues were attached to each triazolyl-containing peptide resin yielding intermediates 39-41. After Fmoc deprotection, acidolytic cleavage from the solid support, ether precipitation, and oxidative disulfide formation, macrocyclic peptides were isolated via semipreparative RP-HPLC. This gave compounds 9, 11, 13, and 15 as white solids.

### EXAMPLE 1

### General Procedures of Peptide Synthesis

All peptide bearing a C-terminal carboxy acid were assembled on a TentaGel S AC resin (Rapp Polymere) or a 2-chlorotrityl chloride resin (Iris Biotech) preloaded with Fmoc-L-Asp(tBu). Corresponding peptide amides were assembled on AmphiSpheres 40 RAM resin (Agilent).

Loading of 2-chlorotrityl chloride resin was performed manually as follows: A solution of 103 mg Fmoc-L-Asp(tBu)-OH (0.25 mmol) and 34 μL N,N-diisopropylethylamine (DIEA, 1 mmol) in a minimal amount of dichloromethane (DCM) was added to the resin. The resulting mixture was shaken for 2 h at ambient temperature. The solution was removed by filtration and the loaded resin was washed with DCM/methanol/DIEA (17:2:1; 3×), DCM (3×), dimethylformamide (DMF, 3×) and DCM (3×).

Canonical amino acids were attached by double or triple coupling using 4 eq of the corresponding amino acid, 3.9 eq of 2-(1H-benzotriazol-1-yl)-1,1,3,3-tetramethyluronoium hexafluorophos [symbol] phate (HBTU) and 8 eq of DIEA, or, in case of cysteine, 3-4 eq of 2,4,6-trimethylpyridine (collidine). Arginine and cysteine were coupled using a two-step microwave program: 1. RT, 0 W, 25 min; 2. 75° C., 25 W, 0.5 min (Arg) and 1. RT, 0 W, 2. min; 2. 50° C., 25 W, 4 min (Cys), respectively. All other amino acids were coupled using a standard microwave program: 75° C., 21 W, 5 min.

Non-natural azide-bearing building blocks Fmoc-L-Aza-OH and Fmoc-L-Aha-OH were attached via double coupling using 2 eq of the corresponding amino acid, 1.9 eq HATU, and 4 eq DIEA and a two-step microwave program (1. 60° C., 30 W, 45 min, 2. 75° C. 20 W, 5 min).

Fmoc deprotection was achieved in two steps by reaction with 20% piperidine in DMF at 75° C., 42 W for 0.5 min (initial deprotection) followed by a second deprotection step with 20% piperidine in DMF at 75° C., 42 W for 3 min.

Cleavage of peptides from the solid support and removal of side-chain protecting groups was achieved via acidolysis using a standard cleavage cocktail consisting of trifluoroacetic acid (TFA)/H2O/anisole/triethylsilane (TES) (47:1:1:1, v:v:v:v) and DTT to suppress unwanted oxidation. The resulting reaction mixture was shaken for 3 h at RT followed by precipitation and subsequent washing (3×) with methyl tertiary butyl ether (MTBE) to yield crude linear peptides.

Air-mediated oxidative macrocyclization of the crude peptides was conducted in 100 mM (NH₄)₂CO₃ aq (1 mg/mL, pH=8.4) and monitored by analytical RP-HPLC. After complete conversion (1-7 days) the solvent was removed by freeze-drying to yield the crude peptides. To suppress unwanted saponification of the methyl ester of compounds 5, 6, 13, and 14, a 100 mM (NH₄)₂CO₃ aq buffer (pH 7.7) was used (FIG. 2).

### EXAMPLE 2

### Triazolyl-containing peptides 1-8, 10, 12, 14, and 16

Azide-bearing peptide resins 23, 24, and 25 were assembled on a TentaGel S AC-Asp(t-Bu) Fmoc resin (loading: 0.21 mmol/g) according to the described procedures on a 0.25 mmol scale, washed (3× with DCM and 3× with ether) and dried in an exsiccator. FIG. 1 shows details.

On-support copper(I)-catalyzed azide-alkyne cycloaddition (CuAAC) was conducted with 0.05 mmol of intermediates 23, 24, and 25 and the respective alkyne component (propargylamine, methyl propiolate, or phenylacetylene) according to the scheme laid out in FIG. 2C. A solution of 5 eq alkyne, 1 eq copper(II) sulfate pentahydrate (CuSO₄. 5H₂O), 1 eq sodium ascorbate (NaAsc) and 8 eq DIEA in 5 mL argon-flushed DMF was added to the peptide resin and shaken at ambient temperature overnight. Then, the solution was removed by filtration and the peptide resin was washed with methanol (3×), 0.5% sodium diethyldithiocarbamate in DMF (w/v, 3×), DMF (3×) and DCM (3×) yielding intermediates 27-35.

After Fmoc deprotection, acidolytic cleavage from the solid support, ether precipitation, and oxidative disulfide formation, macrocyclic peptides were isolated via semi-preparative RP-HPLC. This gave compounds 108, 10, 12, 14, and 16 as white solids in the following yields. 1: 13.5 mg (17%); 2: 14.9 mg (18.6%); 3: 9.3 mg (11.6%); 4: 6.3 mg (7.8%); 5: 2.4 mg (3%); 6: 2 mg (2.5%); 7: 0.5 mg (0.6%) of 7; 8: 5.1 mg (6.2%); 10: 4.6 mg (5.8%); 12: 4 mg (5%); 14: 4 mg (4.9%); 16: 2 mg (2.4%).

### EXAMPLE 3

### Triazolyl-containing peptides 9, 11, 13, and 15

Azide-bearing peptide resin 26 was assembled on a TentaGel S AC-Asp(t-Bu) Fmoc resin (loading: 0.21 mmol/g) according to the described procedures on a 0.25 mmol scale, washed (3× with DCM and 3× with ether) and dried in an exsiccator.

On-support copper(I)-catalyzed azide-alkyne cycloaddition (CuAAC) was conducted with 0.05 mmol of intermediates 26 and the respective alkyne component (N-Boc-propargylamine, methyl propiolate, or phenylacetylene) according to the scheme laid out in FIG. 2D. A solution of 5 eq alkyne, 1 eq copper(II) sulfate pentahydrate (CuSO₄. 5H₂O), 1 eq sodium ascorbate (NaAsc) and 8 eq DIEA in 5 mL argon-flushed DMF was added to the peptide resin and shaken at ambient temperature overnight. Then, the solution was removed by filtration and the peptide resin was washed with methanol (3×), 0.5% sodium diethyldithiocarbamate in DMF (w/v, 3×), DMF (3×) and DCM (3×) yielding intermediates 36-38.

The Fmoc protecting group was removed according to the described procedure. The remaining N-terminal residues were attached to each triazolyl-containing peptide resin yielding intermediates 39□41. After Fmoc deprotection, acidolytic cleavage from the solid support, ether precipitation, and oxidative disulfide formation, macrocyclic peptides were isolated via semi-preparative RP-HPLC. This gave compounds 9, 11, 13, and 15 as white solids in the following yields. 9: 16.8 mg (21.2%); 11: 6.6 mg (8.2%); 13: 4.1 mg (5.1%); 15: 4.2 mg (5.1%).

### EXAMPLE 4

### Triazolyl-Containing Peptide 42

Inhibitor 42 was synthesized following a strategy similar to that described for compounds 9, 11, 13, and 15. First peptide-resin intermediate Fmoc-Aza-Cys(Trt)-His(Trt)-Pro-Asp(tBu)-resin was assembled on a 2-chlorotrityl chloride resin preloaded with Fmoc-Asp(tBu) (loading: 0.23 mmol/g) on a 0.05 mmol scale. Then, a solution of 39 mg N-Boc-propargylamine (0.25 mmol), 14 mg CuSO₄.5H₂O (0.05 mmol), 10 mg NaAsc (0.05 mmol) and 70 μL DIEA (0.4 mmol) in 5 mL argon-flushed DMF were added to the peptide resin and shaken at ambient temperature overnight. The solution was removed by filtration and the peptide resin was washed with methanol (3×), 0.5% sodium diethyldithiocarbamate in DMF (w/v, 3×), DMF (3×) and DCM (3×).

The Fmoc protecting group was removed according to the described procedure. The remaining N-terminal residues were attached to the triazolyl-containing peptide resin. After Fmoc deprotection, acidolytic cleavage from the solid support, ether precipitation, and oxidative disulfide formation, macrocyclic peptide 42 was isolated via semi-preparative RP-HPLC. This gave 0.9 mg (1.2%) of pure 42 as a white solid.

### EXAMPLE 5

### Peptides 17-22, and 43

Linear precursors were synthesized on a 2-chlorotrityl chloride resin preloaded with Fmoc-Asp(tBu) (loading: 0.23 mmol/g) on a 0.05 mmol scale. After acidolytic cleavage from the solid support, ether precipitation, and oxidative disulfide formation, macrocyclic peptides were isolated via semi-preparative RP-HPLC. This gave compounds 17-22, and 43 as white solids in the following yields. 17: 3 mg (3.9%); 18: 6.2 mg (7.9%); 19: 17.4 mg (23.9%); 20: 11.2 mg (15.1%); 21: 23.2 mg (31%); 22: 9.2 mg (12.1%); 43: 0.9 mg (1.2%).

### EXAMPLE 6

### Inhibitor 44

The linear precursor of 44 was synthesized on an AmphiSpheres 40 RAM resin (0.44 mmol/g) on a 0.1 mmol scale. After acidolytic cleavage from the solid support, ether precipitation, and oxidative disulfide formation, the macrocyclic peptide was isolated via semi-preparative RP-HPLC. This gave 10.5 mg (7.8%) of 44 as a white solid.

### EXAMPLE 7

### In Vitro Assays

Enzymes.

Recombinant production, autocatalytic activation and purification of matriptase were conducted as previously described. Bovine trypsin was purchased from Sigma-Aldrich. Active-site titration and determination of Michaelis-Menten constants K_{M} for the chromogenic substrate Boc-QAR-pNA was performed as reported earlier.

Data Collection and Analysis.

All experiments were performed in triplicate. Active-site titrated matriptase (final concentration: [E]=0.9 nM) or trypsin (final concentration: [E]=0.5 nM) were incubated with the respective inhibitor in different concentrations [I] at pH 7.6 or 8.5 for 30 min. Then, the chromogenic substrate Boc-QAR-pNA (final concentration: [S]=250 μM) was added. The residual proteolytic activity (v/v₀) was determined by monitoring the absorption of the corresponding samples in 96-well plates (NUNC, round bottom, clear) at 405 nm in 60 sec intervals over 30 min at RT using the Tecan GENios microplate reader. Slopes of the initial reaction velocities (steady-state) were calculated and normalized to the data of the uninhibited enzymatic hydrolysis of Boc-QAR-pNA. The determined v/v₀ values were plotted against the concentration of the inhibitor. The resulting dose-response curves were fitted either by equation 1 or in case of tight-binding inhibition (trypsin: 42; matriptase: 43 at pH 8.5, 44 at pH 7.6 and 8.5) by equation 2 through non-linear regression.

$$\begin{matrix}\frac{v}{{v}_{0}} = {( 1 + \frac{[ I ]}{{IC}_{50}} )}^{- 1} & ( 1 ) \\ \frac{v}{{v}_{0}} = 1 - \frac{( [ E ] + [ I ] + {K}_{i}^{app} ) - \sqrt{{( [ E ] + [ I ] + {K}_{i}^{app} )}^{2} - 4 [ E ] [ I ]}}{2 [ E ]} & ( 2 )\end{matrix}$$

Substrate-independent inhibition constants Ki were calculated from respective IC50 values or apparent (substrate-dependent) inhibition constants _{Ki}^{app} using equations 3 and 4, respectively.

$$\begin{matrix}{K}_{i} = {{IC}_{50} ( 1 + \frac{[ S ]}{{K}_{M}} )}^{- 1} & ( 3 ) \\ {K}_{i} = {{K}_{i}^{app} ( 1 + \frac{[ S ]}{{K}_{M}} )}^{- 1} & ( 4 )\end{matrix}$$

Errors were calculated through propagation of errors (compare Tischler et al.).[17]

Compounds of the invention preferably have a Ki of less than 100 nM, more preferably less than 50 nM or most preferably less than 12 nM, and ideally less than 10 nM, determined as described above.

For the purposes of the present invention, the term “Aryl” denotes a mono- or bicyclic aromatic homo- or heterocycle having 0, 1, 2, 3 or 4 N, O and/or S atoms and 5, 6, 7, 8, 9, or 10 skeleton atoms, which may be unsubstituted or, independently of one another, mono-, or disub-sti-tuted by R^{5′}, R^{5″},

“Aryl” denotes, for example, unsubstituted phenyl, or naphthyl, fur-thermore preferably, for example, phenyl or naphthyl, each of which is mono-, or disubstituted by methyl, ethyl, isopropyl, fluorine, chlorine, bromine, hydroxyl, methoxy, ethoxy, propoxy, nitro, cyano, formyl, acetyl, propionyl, tri-fluoro-methyl, methanesulfonyl, amino, methyl-amino, dimethyl-amino, diethyl-amino, carboxyl, methoxycarbonyl.

“Aryl” furthermore denotes phenyl, o-, m- or p-tolyl, o-, m- or p-ethyl-phenyl, o-, m- or p-propyl-phenyl, o-, m- or p-isopropylphenyl, o-, m- or p-tert-butyl-phenyl, o-, m- or p-hydroxy-phenyl, o-, m- or p-nitro-phenyl, o-, m- or p-amino-phenyl, o-, m- or p-(N-methyl-amino)-phenyl, o-, m- or p-(N-methyl-amino-carbonyl)-phenyl, o-, m- or p-acetamido-phenyl, o-, m- or p-methoxy-phenyl, o-, m- or p-ethoxy-phenyl, o-, m- or p-ethoxy-carbonyl-phenyl, o-, m- or p-(N,N-di-methyl-amino)-phenyl, o-, m- or p-(N,N-di-methyl-aminocarbonyl)-phenyl, o-, m- or p-(N-ethyl-amino)-phenyl, o-, m- or p-(N,N-diethylamino)-phenyl, o-, m- or p-fluoro-phenyl, o-, m- or p-bromo-phenyl, o-, m- or p-chloro-phenyl, o-, m- or p-(methyl-sulfon-amido)-phenyl, o-, m- or p-(methyl-sulfon-yl)-phenyl, further preferably 2,3-, 2,4-, 2,5-, 2,6-, 3,4- or 3,5-di-fluoro-phenyl, 2,3-, 2,4-, 2,5-, 2,6-, 3,4- or 3,5-dichloro-phenyl, 2,3-, 2,4-, 2,5-, 2,6-, 3,4- or 3,5-di-bromo-phenyl, 2,4- or 2,5-dinitrophenyl, 2,5- or 3,4-di-methoxy-phenyl, 3-nitro-4-chloro-phenyl, 3-amino-4-chloro-, 2-amino-3-chloro-, 2-amino-4-chloro-, 2-amino-5-chloro- or 2-amino-6-chloro-phenyl, 2-nitro-4-N,N-di-methyl-amino- or 3-nitro-4-N,N-dimethyl-amino-phenyl, 2,3-diamino-phenyl, p-iodo-phenyl, 4-fluoro-3-chlorophenyl, 2-fluoro-4-bromophenyl, 3-bromo-6-methoxyphenyl, 3-chloro-6-meth-oxy-phenyl, 3-fluoro-4-meth-oxy-phenyl, 3-amino-6-methyl-phenyl.

“Aryl” furthermore preferably denotes 2- or 3-furyl, 2- or 3-thienyl, 1-, 2- or 3-pyrrolyl, 1-, 2,4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 3- or 4-pyridylmethyl, 2-, 3- or 4-pyridylethyl, 2-, 4-, 5- or 6-pyrimidinyl, 2-, 3-, 5-, or 6-pyrazin-1- or 4-yl, furthermore preferably 1,2,3-tri-a-zol-1-, -4- or -5-yl, 1,2,4-triazol-1-, -3- or 5-yl, 1- or 5-tetrazolyl, 1,2,3-oxa-diazol-4- or -5-yl, 1,2,4-oxadi-azol-3- or -5-yl, 1,3,4-oxadiazol-2-yl, 1,3,4-thiadiazol-2- or -5-yl, 1,2,4-thiadiazol-3- or -5-yl, 1,2,3-thiadiazol-4- or -5-yl, 3-or 4-pyri-da-zinyl, 1-, 2-, 3-, 4-, 5-, 6- or 7-indolyl, 2-, 3-, 4-, 5-, 6- or 7-indazolyl, 2-, 3-, 4- or 5-iso-indolyl, 2-, 6, -or 8-purinyl, 1-, 2-, 4- or 5-benzimidazolyl, 1-, 3-, 4-, 5-, 6- or 7-benzo-pyra-zo-lyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 1-, 3-, 4-, 5-, 6-, 7- or 8-isoquinolinyl, 3-, 4-, 5-, 6-, 7- or 8-quino-linyl, 2-, 4-, 5-, 6-, 7- or 8-quinazolinyl, quin-oxalin-2-, 3-, 4- or 5-yl, 4-, 5-, or 6-phthalazinyl, 2-, 3-, 5-, 6-, 7- or 8-2H-benzo-1,4-oxazinyl, each of which is unsubstituted, or mono-, or disubstituted by methyl, ethyl, isopropyl, fluorine, chlorine, bromine, hydroxyl, methoxy, ethoxy, propoxy, nitro, cyano, formyl, acetyl, propionyl, tri-fluoro-methyl, methanesulfonyl, amino, methyl-amino, dimethyl-amino, diethyl-amino, carboxyl, methoxycarbonyl.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed. To the extent a definition of a term set out in a document incorporated herein by reference conflicts with the definition of a term explicitly defined herein, the definition set out herein controls.

The compounds of the present invention are active as inhibitors of matriptase and specifically bind matriptase. More particularly, preferred compounds bind to the serine protease domain of matriptase and inhibit its activity.

It is believed that these compounds will be useful in the prevention or treatment of cancerous conditions where that cancerous condition is exacerbated by the activity of matriptase.

Another use for the compounds of the present invention is to decrease progression of cancerous conditions and the concomitant degradation of the cellular matrix.

The compounds of the present invention are active as inhibitors of serine protease activity of matriptase and specifically bind to the serine protease domain of matriptase or MTSPI. Accordingly, those compounds that contain sites suitable for linking to a solid/gel support may be used ira vitro for affinity chromatography to purify matriptase from a sample or to remove matriptase from a sample using conventional affinity chromatography procedures. These compounds are attached or coupled to an affinity chromatography either directly or through a suitable linker support using conventional methods. See, e. g., Current Protocols in Protein Science, John Wiley & Sons (J. E. Coligan et al., eds, 1997) and Protein Purification Protocols, Humana Press (S. Doonan, ed., 1966) and references therein.

The compounds of the present invention having matriptase or MTSP1 serine protease inhibitory activity are useful in in vitro assays to measure matriptase or MTSP1 activity and the ratio of complexed to uncomplexed matriptase or MTSP1 in a sample. These assays could also be used to monitor matriptase or MTSP1 activity levels in tissue samples, such as from biopsy or to monitor matriptase activities and the ratio of complexed to uncomplexed matriptase for any clinical situation where measurement of matriptase or MTSP1 activity is of assistance. An assay which determines serine protease activity in a sample could be used in combination with an ELISA which determines total amount of matriptase or MTSP1 (whether complexed or uncomplexed) in order to determine the ratio of complexed to uncomplexed matriptase.

Various animal models can be used to evaluate the ability of a compound of the present invention to reduce primary tumor growth or to reduce the occurrence of metastasis.
These models can include genetically altered rodents (transgenic animals), transplantable tumor cells originally derived from rodents or humans and transplanted onto syngenic or immuno-compromised hosts, or they can include specialized models, such as the CAM model described below, designed to evaluate the ability of a compound or compounds to inhibit the growth of blood vessels (angiogensis) which is believed to be essential for tumor growth.

Other models can also be utilized.

Appropriate animal models are chosen to evaluate the in vivo anti-tumor activity of the compounds described in this invention based on a set of relevant criteria. For example, one criterion might be expression of matriptase or MTSP1 and/or matriptase or MTSP1 mRNA by the particular tumor being examined. Two human prostate derived tumors that meet this criterion-are the LnCap and PC-3 cell lines. Another criterion might be that the tumor is derived from a tissue that normally expresses high levels of matriptase or MTSP1.

Human colon cancers meet this criterion. A third criterion might be that growth and/or progression of the tumor is dependent upon processing of a matriptase or MTSP1 substrate (e. g., sc-u-PA). The human epidermoid cancer Hep-3 fits this criterion. Another criterion might be that growth and/or progression of the tumor is dependent on a biological or pathological process that requires matriptase or MTSP1 activity. Another criterion might be that the particular tumor induces expression of matriptase or MTSP1 by surrounding tissue.

Other criteria may also be used to select specific animal models.

Once appropriate tumor cells are selected, compounds to be tested are administered to the animals bearing the selected tumor cells, and subsequent measurements of tumor size and/or metastatic spread are made after a defined period of growth specific to the chosen model.

The CAM model (chick embryo chorioallantoic membrane model), first described by Ossowski, L., J. Cell Biol., 107: 2437-2445 (1988), provides another method for evaluating the anti-tumor and anti-angiogenesis activity of a compound.

Tumor cells of various origins can be placed on 10 day old CAM and allowed to settle overnight. Compounds to be tested can then be injected intravenously as described by Brooks et al., Methods in Molecular Biology, 129: 257-269, (1999). The ability of the compound to inhibit tumor growth or invasion into the CAM is measured 7 days after compound administration.

When used as a model for measuring-the ability of a compound to inhibit angiogensis, a filter disc containing angiogenic factors, such as basic fibroblast growth factor (bFGF) or vascular ediothelial cell growth factor (VEGF), is placed on a 10 day old CAM as described by Brooks et al., Methods in Molecular Biology, 129: 257-269, (1999). After overnight incubation, compounds to be tested are then administered intravenously. The amount of angiogenesis is measured by counting the amount of branching of blood vessels 48 hours after the administration of compound (Methods in Molecular Biology, 129: 257-269, (1999)).

The compounds of the present invention are useful in vivo for treatment of pathologic conditions which would be ameliorated by decreased serine protease activity of matriptase.

It is believed these compounds will be useful in decreasing or inhibiting metastasis, and degradation of the extracellular matrix in tumors and other neoplasms. These compounds will be useful as therapeutic agents in treating conditions characterized by pathological degradation of the extracellular matrix, including those described hereinabove in the Background and Introduction to the Invention.

The present invention includes methods for preventing or treating a condition in a mammal suspected of having a condition which will be attenuated by inhibition of serine protease activity of matriptase or MTSP1 comprising administering to said mammal a therapeutically effective amount of a compound which selectively inhibits serine protease activity of matriptase or a pharmaceutical composition of the present invention.

The compounds of the present invention are administered in vivo, ordinarily in a mammal, preferably in a human. In employing them in vivo, the compounds can be administered to a mammal in a variety of ways, including orally, parenterally, intravenously, subcutaneously, intramuscularly, colonically, rectally, nasally or intraperitoneally, employing a variety of dosage forms.

In practising the methods of the present invention, the compounds of the present invention are administered alone or in combination with one another, or in combination with other therapeutic or in vivo diagnostic agents.

As is apparent to one skilled in the medical art, a “therapeutically effective amount” of the compounds of the present invention will vary depending upon the age, weight and mammalian species treated, the stage of the disease or pathologic condition being treated, the particular compounds employed, the particular mode of administration and the desired effects and the therapeutic indication. Because these factors and their relationship to determining this amount are well known in the medical arts, the determination of therapeutically effective dosage levels, the amount necessary to achieve the desired result of inhibiting matriptase or MTSP1 serine protease activity, will be within the ambit of one skilled in these arts.

Typically, administration of the compounds of the present invention is commenced at lower dosage levels, with dosage levels being increased until the desired effect of inhibiting matriptase activity to the desired extent is achieved, which would define a therapeutically effective amount. For the compounds of the present invention such doses are between about 0.01 mg/kg and about 100 mg/kg body weight, preferably between about 0.01 and about 10 mg/kg body weight.

In addition, the compounds are suitable to treat other conditions including, but not limited to, unstable angina, refractory angina, myocardial infarction, transient ischemic attacks, thrombotic stroke, embolic stroke, disseminated intravascular coagulation including the treatment of septic shock, deep venous thrombosis in the prevention of pulmonary embolism or the treatment of reocclusion or restenosis of reperfused coronary arteries.

Further, these compounds are useful for the treatment or prophylaxis of those diseases which involve the production and/or action of factor Xa/prothrombinase complex. This includes a number of thrombotic and prothrombotic states in which the coagulation cascade is activated which include but are not limited to, deep venous thrombosis, pulmonary embolism, myocardial infarction, stroke, thromboembolic complications of surgery and peripheral arterial occlusion.

In addition to the disease states noted above, other diseases treatable or preventable by the administration of compounds of this invention include, without limitation, occlusive coronary thrombus formation resulting from either thrombolytic therapy or percutaneous transluminal coronary angioplasty, thrombus formation in the venous vasculature, disseminated intravascular coagulopathy, a condition wherein there is rapid consumption of coagulation factors and systemic coagulation which results in the formation of life-threatening thrombi occurring throughout the microvasculature leading to widespread organ failure, hemorrhagic stroke, renal dialysis, blood oxygenation, and cardiac catheterization.

The compounds of the present invention may also be used in combination with other therapeutic or diagnostic agents. In certain preferred embodiments, the compounds of this invention may be coadministered along with other compounds typically prescribed for these conditions according to generally accepted medical practice such as anticoagulant agents, thrombolytic agents, or other antithrombotics, including platelet aggregation inhibitors, tissue plasminogen activators, urokinase, prourokinase, streptokinase, heparin, aspirin, or warfarin.

The compounds of the present invention may act in a synergistic fashion to prevent reocclusion following a successful thrombolytic therapy and/or reduce the time to reperfusion. These compounds may also allow for reduced doses of the thrombolytic agents to be used and therefore minimize potential hemorrhagic side-effects. The compounds of this invention can be utilized in vivo, ordinarily in mammals such as primates, (e. g. humans), sheep, horses, cattle, pigs, dogs, cats, rats and mice, or in vitro.

The compounds of the invention also find utility in a method for inhibiting the coagulation biological samples, which comprises the administration of a compound of the invention.

The compounds of the present invention may also be used in combination with other therapeutic or diagnostic agents. In certain preferred embodiments, the compounds of this invention may be coadministered along with other compounds typically prescribed for these conditions according to generally accepted medical practice such as anticoagulant agents, thrombolytic agents, or other antithrombotics, including platelet aggregation inhibitors, tissue plasminogen activators, urokinase, prourokinase, streptokinase, heparin, aspirin, or warfarin.

The compounds of the present invention may act in a synergistic fashion to prevent reocclusion following a successful thrombolytic therapy and/or reduce the time to reperfusion. These compounds may also allow for reduced doses of the thrombolytic agents to be used and therefore minimize potential hemorrhagic side-effects. The compounds of this invention can be utilized in vivo, ordinarily in mammals such as primates, (e. g. humans), sheep, horses, cattle, pigs, dogs, cats, rats and mice, or in vitro.

An effective quantity of the compound of interest is employed in treatment. The apppropriate dosage for treatment will be clear to one of skill in the art. The dosage of compounds used in accordance with the invention varies depending on the compound and the condition being treated. The age, lean body weight, total weight, body surface area, and clinical condition of the recipient patient; and the experience and judgment of the clinician or practitioner administering the therapy are among the factors affecting the selected dosage.

Other factors include the route of administration the patient, the patient's medical history, the severity of the disease process, and the potency of the particular compound. The dose should be sufficient to ameliorate symptoms or signs of the disease treated without producing unacceptable toxicity to the patient.

Typically, the dosage is administered at least once a day until a therapeutic result is achieved. Preferably, the dosage is administered twice a day, but more or less frequent dosing can be recommended by the clinician. Once a therapeutic result is achieved, the drug can be tapered or discontinued. Occasionally, side effects warrant discontinuation of therapy. In general, an effective amount of the compound is that which provides either subjective relief of symptoms or an objectively identifiable improvement as noted by the clinician or other qualified observer.

### LITERATURE

[1] W. R. Rypniewski, A. Perrakis, C. E. Vorgias, K. S. Wilson, Protein Eng 1994,
[2] A. Schmidt, C. Jelsch, P. Ostergaard, W. Rypniewski, V. S. Lamzin, J Biol Chem 2003, 278, 43357-43362.
[3] T. M. Antalis, T. H. Bugge, Q. Wu, Prog Mol Biol Transl Sci 2011, 99, 1-50.
[4] T. M. Antalis, M. S. Buzza, K. M. Hodge, J. D. Hooper, S. Netzel-Arnett, Biochem J 2010, 428, 325-346.
[5] a) T. H. Bugge, T. M. Antalis, Q. Wu, J Biol Chem 2009, 284, 23177-23181; b) J. D. Hooper, J. A. Clements, J. P. Quigley, T. M. Antalis, J Biol Chem 2001, 276, 857-860; c) S. Netzel-Arnett, J. D. Hooper, R. Szabo, E. L. Madison, J. P. Quigley, T. H. Bugge, T. M. Antalis, Cancer Metastasis Rev 2003, 22, 237-258.
[6] a) M. S. Buzza, S. Netzel-Arnett, T. Shea-Donohue, et al., P Natl Acad Sci USA 2010, 107, 4200-4205; b) K. List, C. C. Haudenschild, R. Szabo, W. J. Chen, S. M. Wahl, W. Swaim, L. H. Engelholm, N. Behrendt, T. H. Bugge, Oncogene 2002, 21, 3765-3779; c) R. Szabo, J. P. Hobson, K. List, A. Molinolo, C. Y. Lin, T. H. Bugge, J Biol Chem 2008, 283, 29495-29504; d) J. Napp, C. Dullin, F. Muller, K. Uhland, J. B. Petri, A. van de Locht, T. Steinmetzer, F. Alves, Int J Cancer 2010, 127, 1958-1974; e) C. Yuan, L. Q. Chen, E. J. Meehan, N. Daly, D. J. Craik, M. D. Huang, J. C. Ngo, Bmc Struct Biol 2011, 11; f) M. D. Oberst, L. Y. Chen, K. Kiyomiya, C. A. Williams, M. S. Lee, M. D. Johnson, R. B. Dickson, C. Y. Lin, Am J Physiol Cell Physiol 2005, 289, C462-470; g) K. List, R. Szabo, A. Molinolo, et al., Gene Dev 2005, 19, 1934-1950; h) K. Uhland, Cell Mol Life Sci 2006, 63, 2968-2978; i) K. List, Future Oncol 2009, 5, 97-104; j) J. M. Milner, A. Patel, R. K. Davidson, et al., Arthritis Rheum-Us 2010, 62, 1955-1966; k) I. Seitz, S. Hess, H. Schulz, et al., Arterioscl Throm Vas 2007, 27, 769-775. [7] a) T. Steinmetzer, A. Schweinitz, A. Sturzebecher, et al., J Med Chem 2006, 49, 4116-4126; b) C. J. Farady, J. Sun, M. R. Darragh, S. M. Miller, C. S. Craik, J Mol Biol 2007, 369, 1041-1051.
[8] M. L. J. Korsinczky, H. J. Schirra, K. J. Rosengren, J. West, B. A. Condie, L. Otvos, M. A. Anderson, D. J. Craik, J Mol Biol 2001, 311, 579-591.
[9] U. Essmann, L. Perera, M. L. Berkowitz, T. Darden, H. Lee, L. G. Pedersen, J Chem Phys 1995, 103, 8577-8593.
[10] a) K. Hilpert, G. Hansen, H. Wessner, R. Volkmer-Engert, W. Hohne, J Biochem 2005, 138, 383-390; b) A. Lesner, A. Legowska, M. Wysocka, K. Rolka, Curr Pharm Design 2011, 17, 4308-4317; c) A. Legowska, D. Debowski, A. Lesner, M. Wysocka, K. Rolka, Bioorgan Med Chem 2009, 17, 3302-3307; d) J. E. Swedberg, S. J. de Veer, K. C. Sit, C. F. Reboul, A. M. Buckle, J. M. Harris, Plos One 2011, 6; e) D. Scarpi, J. D. McBride, R. J. Leatherbarrow, Bioorgan Med Chem 2004, 12, 6045-6052.
[11] a) S. Jiang, P. Li, S. L. Lee, C. Y. Lin, Y. Q. Long, M. D. Johnson, R. B. Dickson, P. P. Roller, Org Lett 2007, 9, 9-12; b) P. Li, S. Jiang, S. L. Lee, C. Y. Lin, M. D. Johnson, R. B. Dickson, C. J. Michejda, P. P. Roller, J Med Chem 2007, 50, 5976-5983; c) Y. Q. Long, S. L. Lee, C. Y. Lin, I. J. Enyedy, S. M. Wang, P. Li, R. B. Dickson, P. P. Roller, Bioorg Med Chem Lett 2001, 11, 2515-2519; d) F. Beliveau, A. Desilets, R. Leduc, Febs J 2009, 276, 2213-2226.
[12] a) J. L. Wike-Hooley, J. Haveman, H. S. Reinhold, Radiother Oncol 1984, 2, 343-366; b) I. F. Tannock, D. Rotin, Cancer Research 1989, 49, 4373-4384; c) P. Montcourrier, I. Silver, R. Farnoud, I. Bird, H. Rochefort, Clin Exp Metastasis 1997, 15, 382-392; d) T. Yoshitomi, Y. Nagasaki, Biointerphases 2012, 7, 7; e) D. Martinez, M. Vermeulen, A. Trevani, et al., J Immunol 2006, 176, 1163-1171.
[13] a) F. Liu, J. E. Park, W. J. Qian, D. Lim, A. Scharow, T. Berg, M. B. Yaffe, K. S. Lee, T. R. Burke, Jr., Chembiochem 2012, 13, 1291-1296; b) F. Liu, J. E. Park, W. J. Qian, D. Lim, A. Scharow, T. Berg, M. B. Yaffe, K. S. Lee, T. R. Burke, Jr., ACS Chem Biol 2012, 7, 805-810.
[14] M. Nazare, H. Matter, D. W. Will, et al., Angew Chem Int Edit 2012, 51, 905-911.
[15] M. Empting, O. Avrutina, R. Meusinger, S. Fabritz, M. Reinwarth, M. Biesalski, S. Voigt, G. Buntkowsky, H. Kolmar, Angew Chem Int Edit 2011, 50, 5207-5211.
[16] a) L. Melendez-Alafort, P. C. Muzzio, A. Rosato, Anticancer Agents Med Chem 2012, 12, 476-499; b) M. Yoshimoto, T. Hayakawa, M. Mutoh, T. Imai, K. Tsuda, S. Kimura, I. O. Umeda, H. Fujii, K. Wakabayashi, J Nucl Med 2012, 53, 765-771; c) A. Kumar, T. Jindal, R. Dutta, R. Kumar, Annals of Nuclear Medicine 2009, 23, 745-751; d) W. B. Cai, K. Chen, Z. B. Li, S. S. Gambhir, X. Y. Chen, Journal of Nuclear Medicine 2007, 48, 1862-1870.
[17] M. Tischler, D. Nasu, M. Empting, et al., Angew Chem Int Edit 2012, 51, 3708-3712.
[18] E. Krieger, K. Joo, J. Lee, J. Lee, S. Raman, J. Thompson, M. Tyka, D. Baker, K. Karplus, Proteins 2009, 77, 114-122.
[19] L. F. Pacios, Comput Chem 1997, 21, 25-34.
[20] a) A. A. Canutescu, A. A. Shelenkov, R. L. Dunbrack, Protein Sci 2003, 12, 2001-2014; b) R. L. Dunbrack, F. E. Cohen, Protein Sci 1997, 6, 1661-1681.

1. Compounds of the formula I 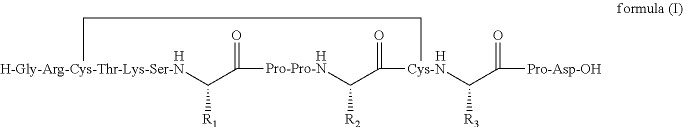
in which
R₁ denotes A or (CH₂)ₙHet,
R₂ denotes A, (CH₂)ₙHet or (CH₂)₃NHC(═NH)NH₂,
R₃ denotes A, (CH₂)ₙHet or (CH₂)ₙAr,
A denotes unbranched or branched alkyl with 1, 2, 3, 4, 5 or 6 C-atoms,
Ar denotes phenyl,
Het denotes furyl, thienyl, pyrrolyl, imidazolyl, pyrazolyl, oxazolyl, isoxazolyl, oxadiazolyl, thiazolyl, triazolyl or tetrazolyl, each of which is unsubstituted or monosubstituted by (CH₂)ₙNH₂, COOH, COOA or Ar,
n denotes 0, 1, 2 or 3,
and pharmaceutically acceptable solvates, salts, tauto [symbol] imers and stereoisomers thereof, including mixtures thereof in all ratios.

2. Compounds according claim 1 in which
Het denotes triazolyl or imidazolyl, which is unsubstituted or monosubstituted by (CH₂)ₙNH₂, COOH, COOA or Ar,
and pharmaceutically acceptable solvates, salts, tauto [symbol] imers and stereoisomers thereof, including mixtures thereof in all ratios.

3. Compound according to Formula (I)
with non-naturally occurring substitutions at positions R₁, R₂ and/or R₃.

4. A mutated SFTI according to claim 1, wherein R₁, R₂ and/or R₃ are selected from C₁-C₄ lower alkyl or arylalkyl.

5. A mutated STFI according to claim 1 selected from the group consisting of

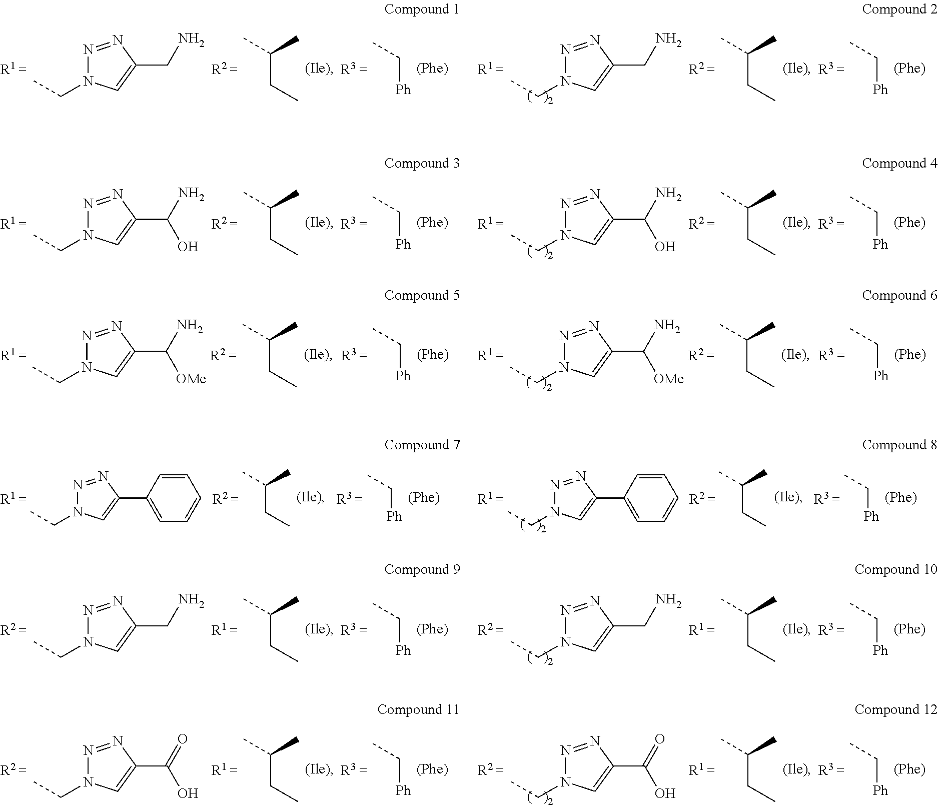

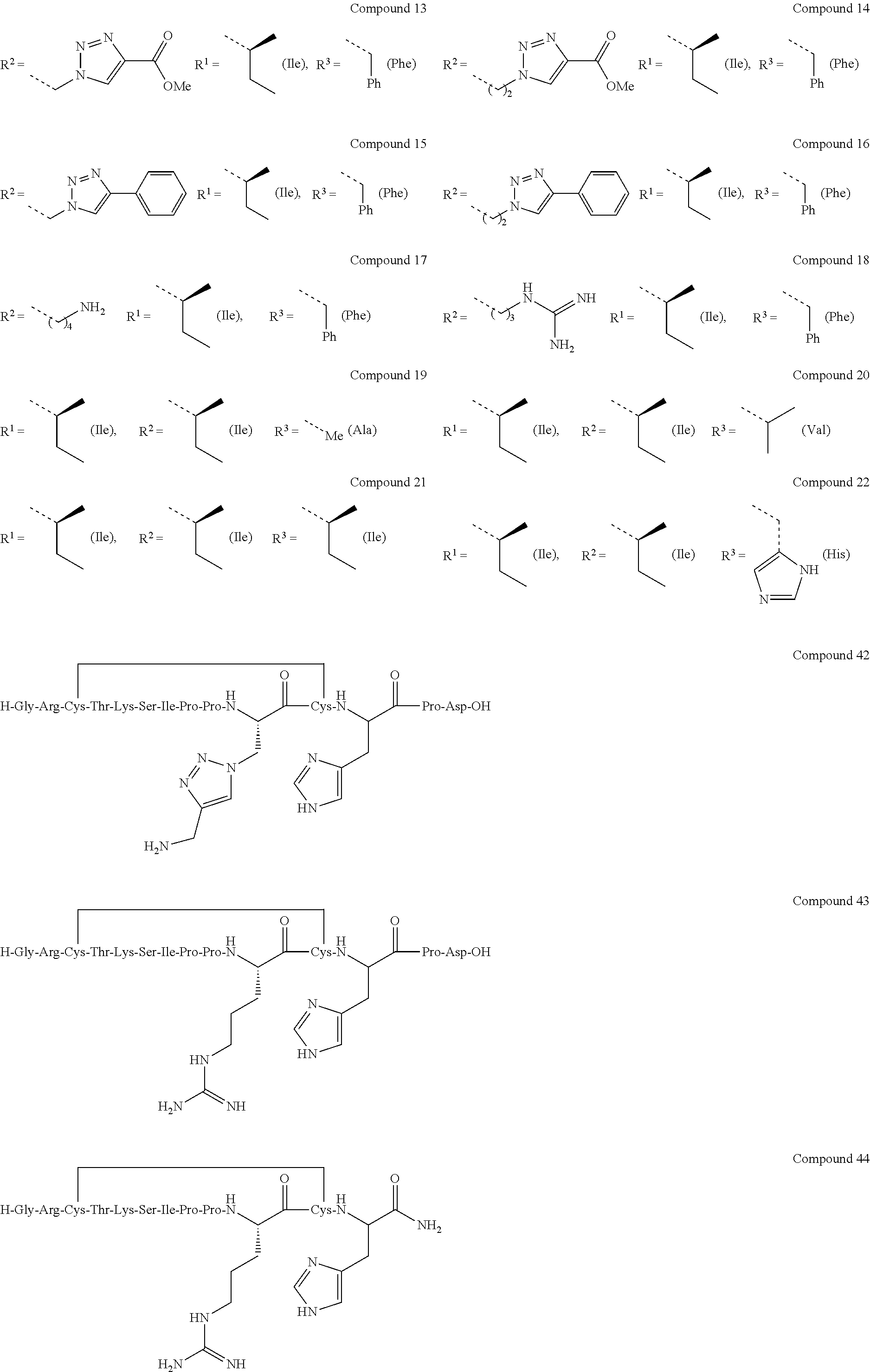

6. A pharmaceutical composition comprising a mutated SFTI of claim 1 and a pharmaceutical acceptable excipient.

7. A method for the treatment of cancer comprising administering to a patient a pharmaceutical composition according to claim 6.

8. A method for the treatment of a matriptase protease dysfunction related disease, wherein the said disease is selected from cancer, chronic obstructive pulmonary disease, a disorder of the peripheral or central nervous system or a cardiovascular disorder, said method comprising administering to a patient a pharmaceutical composition according to claim 6, whereby symptoms of the matriptase dysfunction related disease are ameliorated.

9. A mutated STFI according to claim 1 selected from the group consisting of

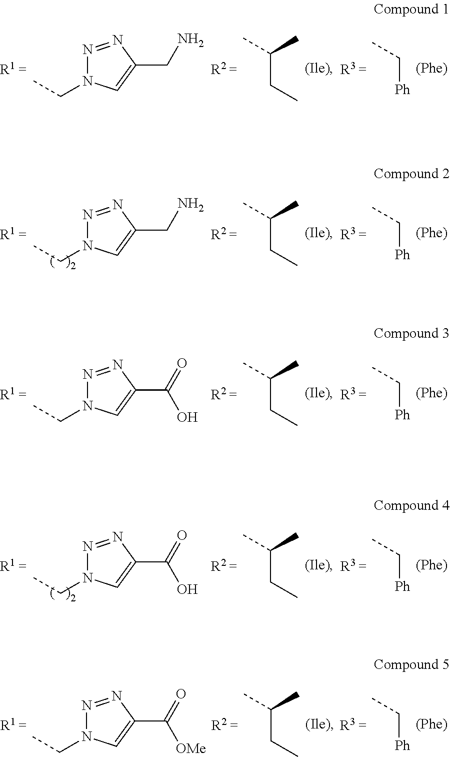

10. A mutated STFI according to claim 1 selected from the group consisting of

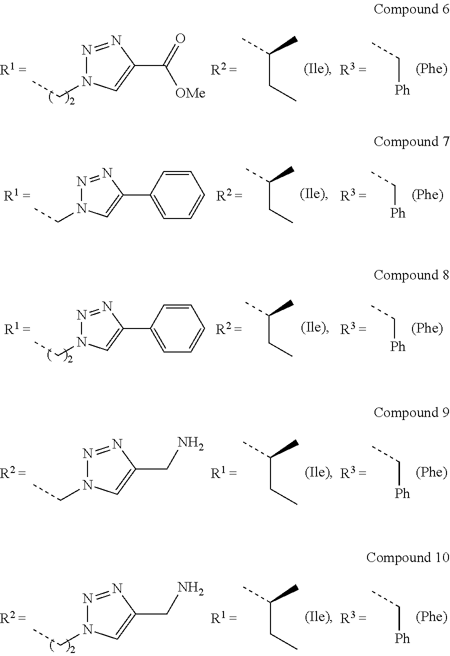

11. A mutated STFI according to claim 1 selected from the group consisting of

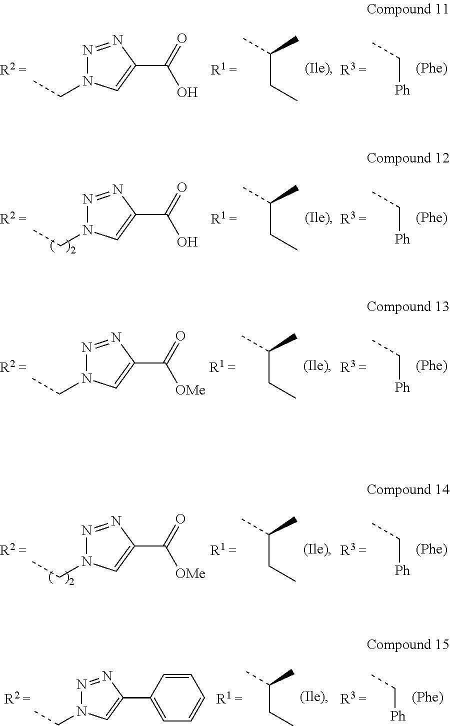

12. A mutated STFI according to claim 1 selected from the group consisting of

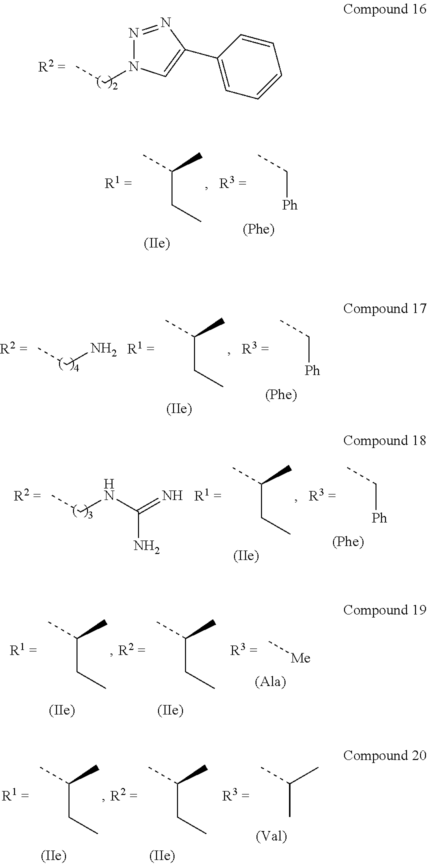

13. A mutated STFI according to claim 1 selected from the group consisting of

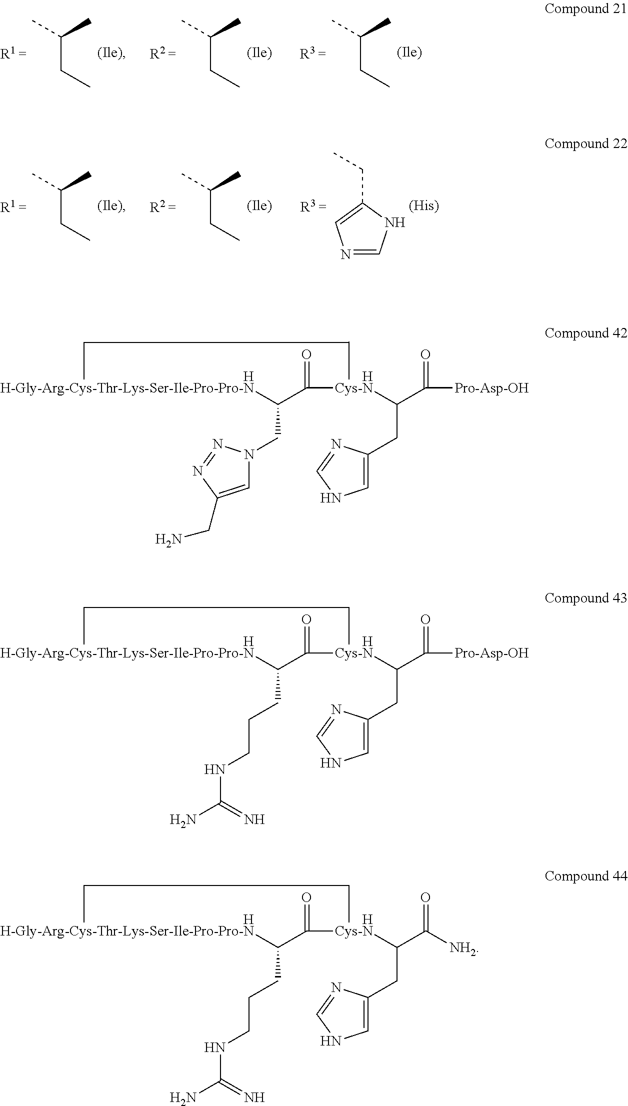

14. A pharmaceutical composition comprising a mutated SFTI of claim 5 and a pharmaceutical acceptable excipient.

15. A method for the treatment of cancer comprising administering to a patient a pharmaceutical composition according to claim 14.

16. A method for the treatment of a matriptase protease dysfunction related disease, wherein said disease is selected from cancer, chronic obstructive pulmonary disease, a disorder of the peripheral or central nervous system or a cardiovascular disorder, said method comprising administering to a patient a pharmaceutical composition according to claim 14, whereby symptoms of the matriptase dysfunction related disease are ameliorated.
